(19) United States
(12) Patent Application Publication
(10) Pub. No.: US 2004/0171004 A1
(43) Pub. Date: Sep. 2, 2004

(54) Chemical compounds

(76) Inventor: John Edward Norris Morten, Cheshire (GB)

Correspondence Address: FISH & RICHARDSON PC, 225 FRANKLIN ST, BOSTON, MA, 02110, US

(21) Appl. No.: 10/474,976

(22) PCT Filed: Apr. 11, 2002
(86) PCT No.: PCT/GB02/01681

Related U.S. Application Data
(60) Provisional application No. 60/284,530, filed on Apr. 19, 2001.

(30) Foreign Application Priority Data
Apr. 17, 2001 (GB) 0109296.4

(51) Int. Cl. C12Q 1/68

(52) U.S. Cl. 435/6

(57) ABSTRACT

This invention relates to polymorphisms in the human SLC10A2 gene and corresponding novel allelic polypeptides encoded thereby. The invention also relates to methods and materials for analysing allelic variation in the SLC10A2 gene, and to the use of SLC10A2 polymorphism in treatment of diseases with SLC10A2 drugs.

This invention relates to polymorphisms in the human ileal sodium-dependent bile acid transporter gene (SLC10A2) and corresponding novel allelic polypeptides encoded thereby. The invention also relates to methods and materials for analysing allelic variation in the SLC10A2 gene, and to the use of SLC10A2 polymorphism in treatment of diseases with SLC10A2 drugs.

Bile acids are synthesised in the liver from cholesterol and secreted into the small intestine, where they are used for the absorption of fat, fat-soluble vitamins, and cholesterol. These bite acids are reabsorbed from the small intestine and resecreted into bile from the liver via the venous portal circulation. This process is extremely efficient with less than 10% of bile acids being excreted via faeces.

The active uptake of bile acids from the small intestine is mediated by a Na⁺-dependent transport system located at the apical brush-border membrane of the ileocyte. In 1995 Wong et al isolated. the human ileal Na⁺/bile acid cotransporter cDNA (gene SLC10A2) and mapped it to chromosome 13q33. Whilst cloning and characterising the gene a dysfunctional missense mutation was identified in a patient diagnosed with Crohn's disease. This mutation involved a single C to T transition, resulting in a proline to serine substitution at amino acid position 290. Oelkers et al found that the mutation did not interfere with protein expression or trafficking to the cell surface, but blocked the transport activity of taurocholate and other bile acids. Inheritance of this mutation was confirmed by single-strand confirmation polymorphism analysis and DNA sequencing.

In 1997 Oelkers et al reported that inherited loss-of-function mutations in SLC10A2 can cause primary bile acid malabsorption (PBAM). Studies of PBAM patients suggested autosomal recessive inheritance, and this was supported by the lack of clinical symptoms in the proband's son. PBAM is involved in a lowered LDL cholesterol level as increased loss of bile acids lowers plasma LDL cholesterol levels due to the diversion of hepatic cholesterol to bile acid synthesis. The proband described in the Oelkers et al paper had a LDL cholesterol level consistently below the 10^{th} percentile. This has been used as a surrogate marker for ileal Na⁺/bile acid co-transporter function.

It is well-known that hyperlipidaemic conditions associated with elevated concentrations of total cholesterol and low-density lipoprotein cholesterol are major risk factors for cardiovascular atherosclerotic disease (for instance “Coronary Heart Disease: Reducing the Risk; a Worldwide View” Assman G., Carmena R. Cullen P. et al; Circulation 1999, 100, 1930-1938 and “Diabetes and Cardiovascular Disease: A Statement for Healthcare Professionals from the American Heart Association” Grundy S, Benjamin I., Burke G., et al; Circulation, 1999, 100, 1134-46). Interfering with the circulation of bile acids within the lumen of the intestinal tracts is found to reduce the level of cholesterol. Previous established therapies to reduce the concentration of cholesterol involve, for instance, treatment with HMG-CoA reductase inhibitors, preferably statins such as simvastatin and fluvastatin, or treatment with bile acid binders, such as resins. Frequently used bile acid binders are for instance cholestyramine and cholestipol. One recently proposed therapy (“Bile Acids and Lipoprotein Metabolism: a Renaissance for Bile Acids in the Post Statin Era” Angelin B, Eriksson M, Rudling M; Current Opinion on Lipidology, 1999, 10, 269-74) involved the treatment with substances with an ileal bile acid transport (IBAT) inhibitory effect.

Re-absorption of bile acid from the gastrointestinal tract is a normal physiological process which mainly takes place in the ileum by the IBAT mechanism. Inhibitors of IBAT can be used in the treatment of hypercholesterolaemia (see for instance “Interaction of bile acids and cholesterol with nonsystemic agents having hypocholesterolaemic properties”, Biochemica et Biophysica Acta, 1210 (1994) 255-287). Thus, suitable compounds having such inhibitory IBAT activity are also useful in the treatment of hyperlipidaemic conditions. Substituted benzothiazepines possessing such IBAT inhibitory activity have been described, see for instance hypolipidaemic benzothiazepine compounds described in WO 93/16055, WO 94/18183, WO 94/18184, WO 96/05188, WO 96/08484, WO 96/16051, WO 97/33882, WO 98/38182, WO 99/35135, WO 98/40375 and EP 0 864 582.

Thus SLC10A2 is a target for cholesterol lowering drugs. Genetic polymorphism in the gene may lead to variation in response to such drugs in the population.

A common polymorphism A171S was published, in 1997 by Oelkers et al. This polymorphism, is found in exon 3, and is present in 28% of individuals in a Caucasian control population of 104 unaffected individuals. It encodes a G to T transversion which results in an alanine to serine substitution in the transporter's third predicted transmembrane domain. Oelkers et al report that this polymorphism does not affect ileal Na⁺/bile acid cotransporter protein expression or taurocholate uptake. This invention relates to further genetic polymorphisms in SLC10A2.

### REFERENCES

Wong, M. H., Oelkers, P., and P. A Dawson. 1995. Identification of a mutation in the ileal sodium-dependent bile acid transporter gene that abolishes transport activity. J. Biol. Chem. 270,27228-27234.

Wong, M. H., Nagesh Rao, P., Pettenati, M. J., and P. A. Dawson. 1996. Localisation of the ileal sodium-bile acid cotransporter gene (SLC10A2) to human chromosome 13q33. Genomics 33, 538-540.

Oelkers, P., Kirby, L. C., Heubi, J. E., and P. A. Dawson. 1997. Primary bile acid malabsorption caused by mutations in the ileal sodium-dependent bile acid transporter gene (SLC10A2). J. Clin. Invest. 99, 1880-1887.

The present invention is based on the discovery of polymorphisms in SLC10A2. In particular, we have found 16 polynucleotide polymorphisms in the SLC10A2 gene, 6 of which lead to changes in the sequence of expressed protein.

According to one aspect of the present invention there is provided a method for the diagnosis of a polymorphism in SLC10A2 in a human, which method comprises determining the sequence of the human at at least one polymorphic position and determining the status of the human by reference to polymorphism in SLC10A2. Preferred polymorphic positions are one or more of the following positions:

positions 582,664,727,792,890,1073,1103,1384,1466,1484,1545,1646,1683 and 1765 as defined by the position in SEQ ID NO: 1;

position 1982 as defined by the position in SEQ ID NO: 2;

position 258 as defined by the position in SEQ ID NO: 3; and

positions 65, 98, 159, 290, 296 and 316 as defined by the position in SEQ ID NO: 4.

The term human includes both a human having or suspected of having a SLC10A2 mediated disease and an asymptomatic human who may be tested for predisposition or susceptibility to such disease. At each position the human may be homozygous for an allele or the human may be a heterozygote.

The term polymorphism includes single nucleotide substitution, nucleotide insertion and nucleotide deletion which in the case of insertion and deletion includes insertion or deletion of one or more nucleotides at a position of a gene and corresponding alterations in expressed protein.

In one embodiment of the invention preferably the method for diagnosis described herein is one in which the polymorphism in SLC10A2 is any one of the following:

| | | | |
|---|---|---|---|
| | | Allele in | |
| | | SEQ ID | Variant |
| Ref. Sequence | Position | NO x | Allele |

| | | | |
|---|---|---|---|
| SEQ ID NO: 1 | 582 | C | G |
| SEQ ID NO: 1 | 664 | T | C |
| SEQ ID NO: 1 | 727 | C | T |
| SEQ ID NO: 1 | 792 | C | T |
| SEQ ID NO: 1 | 890 | G | A |
| SEQ ID NO: 1 | 1073 | G | A |
| SEQ ID NO: 1 | 1103 | C | T |
| SEQ ID NO: 1 | 1384 | G | T |
| SEQ ID NO: 1 | 1466 | C | T |
| SEQ ID NO: 1 | 1484 | T | C |
| SEQ ID NO: 1 | 1545 | G | A |
| SEQ ID NO: 1 | 1646 | A | T |
| SEQ ID NO: 1 | 1683 | T | C |
| SEQ ID NO: 1 | 1765 | T | C |
| SEQ ID NO: 2 | 1982 | T | C |
| SEQ ID NO: 3 | 258 | G | A |
| SEQ ID NO: 4 | 65 | P | L |
| SEQ ID NO: 4 | 98 | V | I |
| SEQ ID NO: 4 | 159 | V | I |
| SEQ ID NO: 4 | 290 | P | S |
| SEQ ID NO: 4 | 296 | F | L |
| SEQ ID NO: 4 | 316 | G | E |

The method for diagnosis is preferably one in which the polynucleotide sequence is determined by a method selected from amplification refractory mutation system, restriction fragment length polymorphism and primer extension.

The status of the individual may be determined by reference to allelic variation at any 1, 2, 3, 4, 5, 6, 7, 8, 9, 10, 11, 12, 13, 14, 15, 16, 17, 18 or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

TABLE 1
Mutation Detection Techniques

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| ELISA | Enzyme Linked ImmunoSorbent Assay |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

General: DNA sequencing, Sequencing by hybridisation

Scanning: PTT*, SSCP, DGGE, TGGE, Cleavase, Heteroduplex analysis, CMC, Enzymatic mismatch cleavage

*Note: not useful for detection of promoter polymorphisms.

Hybridisation Based

Solid phase hybridisation: Dot blots, MASDA, Reverse dot blots, Oligonucleotide arrays (DNA Chips).

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B 1 (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay

Table 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Hybridisation protection assay, Mass spectrometry

Table 3—Further Amplification Methods

SSR, NASBA, LCR, SDA, b-DNA

Table 4—Protein Variation Detection Methods

Immunoassay

Immunohistology

Peptide sequencing

Preferred mutation detection techniques include ARMS™, ALEX™, COPS, Taqman, Molecular Beacons, RFLP, and restriction site based PCR and FRET techniques. Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom.

Particularly preferred methods include ARMS™ and RFLP based methods. ARMS™ is an especially preferred method.

In a further aspect, the diagnostic methods of the invention are used to assess the pharmacogenetics of a SLC10A2 drug.

Assays, for example reporter-based assays, may be devised to detect whether one or more of the above polymorphisms affect transcription levels and/or message stability.

Individuals who carry particular allelic variants of the SLC10A2 gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by SLC10A2. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the SLC10A2 gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human SLC10A2 gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 bases of the human SLC10A2 gene and comprising an allelic variant selected from any one of the following:

| | | |
|---|---|---|
| | | Variant |
| Ref. Sequence | Position | Allele |

| | | |
|---|---|---|
| SEQ ID NO: 1 | 582 | G |
| SEQ ID NO: 1 | 664 | C |
| SEQ ID NO: 1 | 727 | T |
| SEQ ID NO: 1 | 792 | T |
| SEQ ID NO: 1 | 890 | A |
| SEQ ID NO: 1 | 1073 | A |
| SEQ ID NO: 1 | 1103 | T |
| SEQ ID NO: 1 | 1384 | T |
| SEQ ID NO: 1 | 1466 | T |
| SEQ ID NO: 1 | 1484 | C |
| SEQ ID NO: 1 | 1545 | A |
| SEQ ID NO: 1 | 1646 | T |
| SEQ ID NO: 1 | 1683 | C |
| SEQ ID NO: 1 | 1765 | C |
| SEQ ID NO: 2 | 1982 | C |
| SEQ ID NO: 3 | 258 | A |

According to another aspect of the present invention there is provided a human SLC10A2 gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a SLC10A2 gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a SLC10A2 gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a SLC10A2 gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms at 1765 in SEQ ID NO 1 and at 258 in SEQ ID NO 3 because of their relatively high frequencies (see below). The SLC10A2 gene has been mapped to human chromosome 13q33 (Wong et al, Genomics 33: 538-540, 1996).

Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse JF, London J, Stansbie D, Humphries S E, and Gudnason V; and Variation at the von willebrand factor (vWF) gene locus is associated with plasma vWF:Ag levels: identification of three novel single nucleotide polymorphisms in the vWP gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a SLC10A2 drug in which the method comprises:

i) diagnosis of a polymorphism in SLC10A2 in the human, which diagnosis preferably comprises determining the sequence at one or more of the following positions:

positions 582,664,727,792,890,1073,1103,1384,1466,1484,1545,1646,1683 and 1765 as defined by the position in SEQ ID NO: 1;

position 1982 as defined by the position in SEQ ID NO: 2;

position 258 as defined by the position in SEQ ID NO: 3; and

positions 65, 98, 159, 290, 296 and 316 as defined by the position in SEQ ID NO: 4.

and determining the status of the human by reference to polymorphism in the SLC10A2 gene; and

ii) administering an effective amount of the drug.

Preferably determination of the status of the human is clinically useful. Examples of clinical usefulness include deciding which drug or drugs to administer and/or in deciding on the effective amount of the drug or drugs. The term “SLC10A2 drug” means either that interaction with SLC10A2 in humans is an aspect of a drug exerting its pharmceutical effect in man or that SCL10A2 is involved in the biological pathway through which drug exerts its pharmceutical effect in man or that the drug is transported by SLC10A2. For example, substituted benzothiazepines possessing SLC10A2 inhibitory activity have been described, see for instance hypolipidaemic benzothiazepine compounds described in WO 93/16055, WO 94/18183, WO 94/18184, WO 96/05188, WO 96/08484, WO 96/16051, WO 97/33882, WO 98/38182, WO 99/35135, WO 98/40375 and EP 0 864 582.

According to another aspect of the present invention there is provided an allelic variant of human SLC10A2 polypeptide comprising at least one of the following:

| | |
|---|---|
| Position | Allelic variant |

| | | |
|---|---|---|
| SEQ ID NO: 4 | 65 | L |
| SEQ ID NO: 4 | 98 | I |
| SEQ ID NO: 4 | 159 | I |
| SEQ ID NO: 4 | 290 | S |
| SEQ ID NO: 4 | 296 | L |
| SEQ ID NO: 4 | 316 | E |

or a fragment thereof comprising at least 10 amino acids provided that the fragment comprises at least one allelic variant.

Fragments of polypeptide are at least 10 amino acids, more preferably at least 15 amino acids, more preferably at least 20 amino acids.

According to another aspect of the present invention there is provided an antibody specific for an allelic variant of human SLC10A2 polypeptide as described herein.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of SLC10A2 with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples. All temperatures are in degrees Celsius.

In the Examples below, unless otherwise stated, the following methodology and materials have been applied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes 1-3, edited by F M Asubel, R Brent and R E Kingston; published by John Wiley, 1998.

Electropherograms were obtained in a standard manner: data was collected by ABI377 data collection software and the wave form generated by ABI Prism sequencing analysis (2.1.2).

### EXAMPLE 1

Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2^{nd} Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide primers and annealing temperatures set out below. The extension temperature was 72° and denaturation temperature 94°. Generally 50 ng of genomic DNA was used in each reaction and subjected to 35 cycles of PCR. Where described below, the primary fragment was diluted 1/100 and two microlitres were used as template for amplification of secondary fragments. PCR was performed in two stages (primary fragment then secondary fragment) to ensure specific amplification of the desired target sequence.

2 Polymorphisms in SLC10A2

The following polymorphisms were discovered in SLC10A2 using both cDNA and genomic DNA templates.

| | | | | | |
|---|---|---|---|---|---|
| | | Published | Novel | | |
| Ref. Sequence | Position | Allele | Allele | Frequency | Comment |

| | | | | | | |
|---|---|---|---|---|---|---|
| SEQ ID NO: 1 | 582 | C | G | 1/56 | 5′UTR | |
| SEQ ID NO: 1 | 664 | T | C | 1/56 | silent | |
| SEQ ID NO: 1 | 727 | C | T | 1/56 | silent | |
| SEQ ID NO: 1 | 792 | C | T | 1/46 | P65L | SEQ ID NO: 4 |
| SEQ ID NO: 1 | 890 | G | A | 3/58 | V98I | SEQ ID NO: 4 |
| SEQ ID NO: 1 | 1073 | G | A | 1/56 | V159I | SEQ ID NO: 4 |
| SEQ ID NO: 1 | 1103 | C | T | 1/56 | silent | |
| SEQ ID NO: 1 | 1384 | G | T | 1/56 | silent | |
| SEQ ID NO: 1 | 1466 | C | T | 1/56 | P290S | SEQ ID NO: 4 |
| SEQ ID NO: 1 | 1484 | T | C | 1/56 | F296L | SEQ ID NO: 4 |
| SEQ ID NO: 1 | 1545 | G | A | 1/56 | G316E | SEQ ID NO: 4 |
| SEQ ID NO: 1 | 1646 | A | T | 1/56 | 3′UTR | |
| SEQ ID NO: 1 | 1683 | T | C | 1/56 | 3′UTR | |
| SEQ ID NO: 1 | 1765 | T | C | 30/50 | 3′UTR | |
| SEQ ID NO: 2 | 1982 | T | C | 1/56 | intron 1 | |
| SEQ ID NO: 3 | 258 | G | A | 16/56 | intron 5 | |

1 A method for the diagnosis of a polymorphism in SLC10A2 in a human, which method comprises determining the sequence of the human at one or more of the following positions:
positions 582,664,727,792,890,1073,1103,1384,1466,1484,1545,1646,1683 and 1765 as defined by the position in SEQ ID NO: 1;
position 1982 as defined by the position in SEQ ID NO: 2;
position 258 as defined by the position in SEQ ID NO: 3; and
positions 65, 98, 159, 290, 296 and 316 as defined by the position in SEQ ID NO: 4.
and determining the status of the human by reference to polymorphism in SLC10A2.

2 The method for diagnosis according to claim one which the polymorphism in SLC10A2 is any one of the following:

| | | | |
|---|---|---|---|
| | | Allele in | |
| | | SEQ ID | Variant |
| Ref. Sequence | Position | NO x | Allele |
| | | | |
|---|---|---|---|
| SEQ ID NO: 1 | 582 | C | G |
| SEQ ID NO: 1 | 664 | T | C |
| SEQ ID NO: 1 | 727 | C | T |
| SEQ ID NO: 1 | 792 | C | T |
| SEQ ID NO: 1 | 890 | G | A |
| SEQ ID NO: 1 | 1073 | G | A |
| SEQ ID NO: 1 | 1103 | C | T |
| SEQ ID NO: 1 | 1384 | G | T |
| SEQ ID NO: 1 | 1466 | C | T |
| SEQ ID NO: 1 | 1484 | T | C |
| SEQ ID NO: 1 | 1545 | G | A |
| SEQ ID NO: 1 | 1646 | A | T |
| SEQ ID NO: 1 | 1683 | T | C |
| SEQ ID NO: 1 | 1765 | T | C |
| SEQ ID NO: 2 | 1982 | T | C |
| SEQ ID NO: 3 | 258 | G | A |
| SEQ ID NO: 4 | 65 | P | L |
| SEQ ID NO: 4 | 98 | V | I |
| SEQ ID NO: 4 | 159 | V | I |
| SEQ ID NO: 4 | 290 | P | S |
| SEQ ID NO: 4 | 296 | F | L |
| SEQ ID NO: 4 | 316 | G | E |

3 Use of a diagnostic method as defined in claim 1 to assess the pharmacogenetics of a drug acting at SLC10A2.

4 A polynucleotide comprising at least 20 bases of the human SLC10A2 gene and comprising an allelic variant selected from any one of the following:

| | | |
|---|---|---|
| | | Variant |
| Ref. Sequence | Position | Allele |
| | | |
|---|---|---|
| SEQ ID NO: 1 | 582 | G |
| SEQ ID NO: 1 | 664 | C |
| SEQ ID NO: 1 | 727 | T |
| SEQ ID NO: 1 | 792 | T |
| SEQ ID NO: 1 | 890 | A |
| SEQ ID NO: 1 | 1073 | A |
| SEQ ID NO: 1 | 1103 | T |
| SEQ ID NO: 1 | 1384 | T |
| SEQ ID NO: 1 | 1466 | T |
| SEQ ID NO: 1 | 1484 | C |
| SEQ ID NO: 1 | 1545 | A |
| SEQ ID NO: 1 | 1646 | T |
| SEQ ID NO: 1 | 1683 | C |
| SEQ ID NO: 1 | 1765 | C |
| SEQ ID NO: 2 | 1982 | C |
| SEQ ID NO: 3 | 258 | A |

5 A nucleotide primer which can detect a polymorphism as defined in claim 1.

6 An allele specific primer capable of detecting a SLC10A2 gene polymorphism as defined in claim 1.

7 An allele-specific oligonucleotide probe capable of detecting a SLC10A2 gene polymorphism as defined in claim 1.

8 Use of a SLC10A2 gene polymorphism as defined in claim 1 as a genetic marker in a linkage study.

9 A method of treating a human in need of treatment with a SLC10A2 drug in which the method comprises:
i) diagnosis of a polymorphism in SLC10A2 in the human, which diagnosis preferably comprises determining the sequence at one or more of the following positions:
positions 582,664,727,792,890,1073,1103,1384,1466,1484,1545,1646,1683 and 1765 as defined by the position in SEQ ID NO: 1;
position 1982 as defined by the position in SEQ ID NO: 2;
position 258 as defined by the position in SEQ ID NO: 3; and
positions 65, 98, 159, 290, 296 and 316 as defined by the position in SEQ ID NO: 4.
and determining the status of the human by reference to polymorphism in SLC10A2; and
ii) administering an effective amount of the drug.

10 An allelic variant of human SLC10A2 polypeptide comprising at least one of the following:

| | |
|---|---|
| Position | Allelic variant |
| | | |
|---|---|---|
| SEQ ID NO: 4 | 65 | L |
| SEQ ID NO: 4 | 98 | I |
| SEQ ID NO: 4 | 159 | I |
| SEQ ID NO: 4 | 290 | S |
| SEQ ID NO: 4 | 296 | L |
| SEQ ID NO: 4 | 316 | E |

or a fragment thereof comprising at least 10 amino acids provided that the fragment comprises at least one allelic variant.

11 An antibody specific for an allelic variant of human SLC10A2 polypeptide as defined in claim 10.
